(19) United States
(12) Patent Application Publication
(10) Pub. No.: US 2016/0058379 A1
(43) Pub. Date: Mar. 3, 2016

(54) Animal of Equidae Family Band or Collar for Health & Vital Signs Monitoring, Alert and Diagnosis

(71) Applicant: PetPlace Ltd., Ramat Hasharon (IL)

(72) Inventors: Avi MENKES, Ramat Hasharon (IL); Asaf DAGAN, Ramat Hasharon (IL)

(21) Appl. No.: 14/468,356

(22) Filed: Aug. 26, 2014

(51) Int. Cl. A61B 5/00; A61B 7/00; A61B 5/103; A61B 5/0205; A61B 5/11

(52) U.S. Cl. CPC A61B 5/6831; A61B 5/02055; A61B 5/1118; A61B 5/1123; A61B 5/1112; A61B 5/1114; A61B 5/6822; A61B 5/6828; A61B 5/7282; A61B 5/746; A61B 5/1036; A61B 7/008; A61B 5/4866; A61B 5/747; A61B 5/0008; A61B 5/01

(57) ABSTRACT

A system for monitoring vital signs of an animal such as a horse comprises a band on leg(s) or a neck, an accelerometer configured to measure at least one of resting patterns, activity patterns, movement patterns, position patterns, lameness, kicking, stomping, lifting leg, pawing, and a non-accelerometer sensor configured to measure at least one of the following non-accelerometer-measured bioparameters of the animal: temperature, pulse rate, respiration rate. One or more processors are configured to receive sensor output data and reference data concerning the measured bioparameters of for example a horse, or of a population of the horse, and determine a suspicion of a specific medical condition by: (i) scoring at least two bioparameters and comparing a cumulative score to a threshold cumulative score or to a threshold cumulative range; or (ii) identifying an abnormal pattern. The processor(s) may send an alert if at least one specific medical condition is suspected.

### FIELD AND BACKGROUND OF THE INVENTION

The present invention relates to apparatuses and methods for monitoring vital signs and health of animals, and, more particularly for monitoring the health and vital signs of animals of the Equidae family, such as horses, and doing so using a specially designed band or collar.

When animals are sick they tend by nature to withdraw and hide their symptoms since they feel defenseless. This behavior makes detection of diseases and treatment of the animal significantly more difficult. In addition, certain diseases may develop acutely and deteriorate into life-threatening conditions very quickly. Therefore, early detection may be of paramount medical significance. Yet, most horses are kept in facilities such as a barn, where continuous professional observation is limited or does not exist. For these animals, acute and serious medical conditions may be detected late in the disease process.

Colic, a disease contracted by horses, is defined as abdominal pain. There are a variety of different causes of colic, some of which can prove fatal without surgical intervention. Colic surgery is usually an expensive procedure since it is a major abdominal surgery, often with intensive aftercare. Among domesticated horses, colic is the leading cause of premature death. The incidence of colic in the general horse population has been estimated as 4-20% over the course of their lifetime.

If a horse that contracts colic is not promptly treated, the horse can die within hours. Although it is standard medical practice to check the vital signs of a sick horse, this tends to occur too late, for example because it takes time to summon a veterinarian, and for the veterinarian to reach the horse. Early detection is often not achieved, yet it has a profound impact on prognosis. In fact, the most important factor affecting prognosis is time elapsed since the onset of clinical signs. In addition, it is very important in order to achieve less suffering of the animal and less likelihood of a severe disease, which can develop if detection occurs late.

Monitoring the vital signs of race horses or breeding horses is important for the additional reason that these horses constitute an economic asset and their fitness and soundness affects their value.

There is a compelling need to have an apparatus and method that will provide early detection and diagnosis of animals such as horses or other members of the Equidae family, and to do so accurately and efficiently without interfering with the comfort and behavior of the animal.

### SUMMARY OF THE PRESENT INVENTION

One aspect of the present invention is a system for monitoring vital signs of an animal of a Equidae family, comprising a band having a working surface configured to wrap around one or more of (a) a neck of the animal and (b) at least one leg of the animal; a sensor array comprising at least one sensor element situated along a circumference of the band, the at least one sensor element including at least one of (i) an accelerometer configured to measure at least one accelerometer-measured bioparameter of the animal from among: resting patterns, activity patterns, movement patterns, position patterns, lameness, kicking, stomping, lifting leg, pawing and (ii) a non-accelerometer sensor configured to measure at least one of the following non-accelerometer-measured bioparameters of the animal: temperature, pulse rate, respiration rate; one or more remote or local processors configured to receive (a) sensor output data from the sensor array concerning the measured one or more bioparameters and (b) reference data concerning the measured one or more bioparameters of the animal or of a population of the animal, the one or more remote or local processors configured to determine whether a specific medical condition is suspected by at least one of the following: (i) scoring at least two bioparameters relative to the reference data and comparing a cumulative score of all scored bioparameters to a threshold cumulative score or to a threshold cumulative range; or (ii) identifying an abnormal pattern in at least one bioparameter from among the at least one of (I) the accelerometer-measured bioparameters and (II) the non-accelerometer-measured bioparameters, the one or more remote or local processors configured to send an alert if at least one specific medical condition is suspected.

A further aspect of the present invention is a system for monitoring vital signs of an animal of an Equidae family, comprising a band having a working surface configured to wrap around one or more of (a) a neck of the animal and (b) at least one leg of the animal; one or more accelerometers situated along a circumference of the band and configured to measure at least one bioparameter of the animal from among: resting patterns, activity patterns, movement patterns, position patterns, lameness, kicking, stomping, lifting leg, pawing, one or more remote or local processors configured to receive (a) sensor output data from the one or more accelerometers concerning the measured bioparameters and (b) reference data concerning the measured bioparameters of the animal or of a population of the animal, the one or more remote or local processors configured to determine whether a specific medical condition is suspected by at least one of the following: (i) scoring at least two bioparameters and comparing a cumulative score of all scored bioparameters to a threshold cumulative score or to a threshold cumulative range; or (ii) identifying an abnormal pattern in at least one accelerometer-measured bioparameter, the one or more remote or local processors configured to send an alert if at least one specific medical condition is suspected.

A still further aspect of the present invention is a method of monitoring an animal of an Equidae family to determine a suspicion of a specific medical condition in the animal, comprising providing a band on the animal configured to wrap around one or more of (a) a neck of the animal and (b) at least one leg of the animal, the band including a sensor array comprising at least one sensor element situated along a circumference of the band, the at least one sensor element including at least one of (i) an accelerometer configured to measure at least one accelerometer-measured bioparameter of the animal from among: resting patterns, activity patterns, movement patterns, position patterns, lameness, kicking, stomping, lifting leg, pawing and (ii) a non-accelerometer sensor configured to measure at least one of the following non-accelerometer-measured bioparameters of the animal: temperature, pulse rate, respiration rate; one or more local or remote processors receiving (a) sensor output data from the sensor array concerning the measured one or more bioparameters and (b) reference data concerning the measured one or more bioparameters of the animal or of a population of the animal, the one or more remote or local processors configured to determine whether a specific medical condition is suspected by at least one of the following: (i) scoring at least two bioparameters relative to the reference data and comparing a cumulative score of all scored bioparameters to a threshold cumulative score or to a threshold cumulative range; or (ii) identifying an abnormal pattern in at least one bioparameter from among the at least one of (i) the accelerometer-measured bioparameters and (ii) the non-accelerometer-measured bioparameters; and the one or more remote or local processors sending an alert if at least one specific medical condition is suspected.

These and other features, aspects and advantages of the present invention will become better understood with reference to the following drawings, descriptions and claims.

### BRIEF DESCRIPTION OF THE DRAWINGS

Various embodiments are herein described, by way of example only, with reference to the accompanying drawings, wherein:

FIG. 1 is a top view of an opened band, in accordance with one embodiment of the present invention;

FIG. 2 is a bottom view of the band of FIG. 1, in accordance with one embodiment of the present invention;

FIG. 2A is a bottom view similar to FIG. 2 but having differently shaped acoustic concentrators, in accordance with one embodiment of the present invention;

FIG. 3 is a longitudinal sectional view of an opened band, in accordance with one embodiment of the present invention;

FIG. 3A is a longitudinal sectional view of an opened band of FIG. 2A, in accordance with one embodiment of the present invention;

FIG. 3B is an enlarged sectional view of a portion along the circumference of the band showing a piezoelectric element inside the band, in accordance with one embodiment of the present invention;

FIG. 4A is a vertical sectional view of a cross-shaped acoustic concentrator, in accordance with one embodiment of the present invention;

FIG. 4B is a bottom view of the acoustic concentrator of FIG. 4A, in accordance with one embodiment of the present invention;

FIG. 5A is a vertical sectional view of a solid acoustic concentrator, in accordance with one embodiment of the present invention;

FIG. 5B is a bottom view of the acoustic concentrator of FIG. 5A, in accordance with one embodiment of the present invention;

FIG. 6A is a vertical sectional view of a dot shaped acoustic concentrator, in accordance with one embodiment of the present invention;

FIG. 6B is a bottom view of the acoustic concentrator of FIG. 6A, in accordance with one embodiment of the present invention;

FIG. 7A is a vertical sectional view of a cross-shaped acoustic balancer, in accordance with one embodiment of the present invention;

FIG. 7B is a bottom view of the acoustic balancer of FIG. 7A, in accordance with one embodiment of the present invention;

FIG. 8A is a vertical sectional view of a solid acoustic balancer, in accordance with one embodiment of the present invention;

FIG. 8B is a bottom view of the acoustic balancer of FIG. 8A, in accordance with one embodiment of the present invention;

FIG. 9A is a vertical sectional view of a dot shaped acoustic balancer, in accordance with one embodiment of the present invention;

FIG. 9B is a bottom view of the acoustic balancer of FIG. 9A, in accordance with one embodiment of the present invention; and

FIG. 10 is a high level scheme of a sensor array and associated electronics, the electronics inside a controller, in accordance with one embodiment of the present invention;

FIG. 11 is a schematic of the architecture of an overall system, in accordance with one embodiment of the present invention;

FIG. 12 is a photo of a horse in which a band of the present invention fitted on each of two of the legs of a horse;

FIG. 13 is a collection of three photos of horses showing activity or postures typical for a horse suffering from colic; and

FIG. 14 is a flow chart showing a method, in accordance with one embodiment of the present invention.

### DETAILED DESCRIPTION OF THE INVENTION

The following detailed description is of the best currently contemplated modes of carrying out the invention. The description is not to be taken in a limiting sense, but is made merely for the purpose of illustrating the general principles of the invention, since the scope of the invention is best defined by the appended claims.

The present invention generally provides a system for monitoring vital signs of animals in the family (a term used to describe a biological classification) Equidae, preferably those animals within the genus equus, such as horses, and determining a suspicion of a specific medical condition. A band may have a working surface configured to wrap around a portion of the animal, for example, one or more of (a) a neck of the animal and (b) at least one leg of the animal. A sensor array may comprise at least one sensor element situated along a circumference of the band, the at least one sensor element including at least one of (i) an accelerometer configured to measure at least one accelerometer-measured bioparameter of the animal from among: resting patterns, activity patterns, movement patterns, position patterns, lameness, kicking, stomping, lifting leg, pawing and (ii) a non-accelerometer sensor configured to measure at least one of the following non-accelerometer-measured bioparameters of the animal: temperature, pulse rate, respiration rate. Preferably, the sensor array comprises at least two sensor elements including the accelerometer and at least one non-accelerometer sensor. One or more remote or local processors are configured (i.e. programmed by software or data instructions stored on a non-transitory computer-readable medium) to receive (a) sensor output data from the sensor array concerning the measured one or more bioparameters and (b) reference data concerning the measured one or more bioparameters of the animal or of a population of the animal, the one or more remote or local processors are configured (i.e. by software or data instructions stored on a computer-readable medium) to determine whether a specific medical condition is suspected by at least one of the following: (i) scoring at least two bioparameters relative to the reference data and comparing a cumulative score of all scored bioparameters to a threshold cumulative score or to a threshold cumulative range this second prong wouldn't apply to pulse alone; or (ii) identifying an abnormal pattern in at least one bioparameter from among the at least one of (i) the accelerometer-measured bioparameters and (ii) the non-accelerometer-measured bioparameters (or preferably where the sensor array has both, from among the accelerometer-measured bioparameters and the non-accelerometer-measured bioparameters). The one or more remote or local processors may be configured (i.e. programmed by software or data instructions) to send an alert if at least one specific medical condition is suspected.

For example, the at least one specific medical condition may be one or more of colic, laminitis or lameness. The at least one sensor element may be a piezoelectric sensor, an acoustic sensor, a temperature sensor, a proximity sensor.

The band may comprise a layer of an elastic material and may be configured to measure at least one bioparameter from the following bioparameters: temperature, heart rate, respiration rate, movement and positions. In some versions, At least one acoustic concentrator, in the form of a bump, may project toward the neck or other body portion of the animal from the working surface at the one or more regions along the circumference. In addition, at least one acoustic balancer may project from the rear surface at the one or more regions along the circumference. The at least one acoustic balancer may be situated at a region along the circumference at least partly behind and preferably directly behind the at least one acoustic concentrators.

The band or collar may have sensor elements that can be activated remotely to check vital signs of the animal (such as respiration, pulse, temperature, movement and positions) and a processor that can interpret the results of multiple vital sign readings. The band or collar may also have a two way communication device attached or integrated thereto that can alert the animal's owner, a veterinarian or the authorities, when appropriate, that the animal is suffering from a particular condition or is exhibiting suspicious behavior or movements. This way, a veterinarian can remotely take a particular vital sign measurement when alerted of the data by signalling the processor to actuate a particular sensor element. The sensor elements that may be embedded in the band of the collar may gather data that can be processed on the collar itself or transmitted to a remote terminal, which can be a home computer, a hand-held device, or a main server computer. In order to dramatically improve the system gain, sensitivity and signal to noise ratio (SNR), an elastic layer may absorb noise from friction due to movement of the animal's head.

In contrast to prior art bands or collars for horses, which are typically located under the saddle, or in one case at the underside of the tail, the entire band of the present invention may be situated on the neck or on one or more legs (for example adjacent and above the hoof of the leg), or on both the neck and one or more legs of the animal, for example a horse or other animal of the Equidae family. In contrast to prior art animal bands or collars, which do not measure vital signs, the band of the present invention may measure vital signs of the animal. For example, it may measure, heart rate, respiration rate, temperature, movement, body positions etc. In contrast to the prior art monitoring systems that may utilize multiple sensors, which may generate many alerts over a short period of time, sometimes even simultaneously, a situation that may overload the system, increase its cost or limit its applicability, the system and method of the present invention may avoid these drawbacks not just by sensing a large number of bioparameters of the animal, but also by fusing the sensed data together. As an example, a combination of pulse data (i.e. elevated heart rate) and movement data (that the animal is at rest) and possibly time of day (that it is night time) may lead to early diagnosis of colic, whereas any one of the parameters above alone would not suffice to achieve such early detection. The system of the present invention may combine skin temperature near the hoof area of the horse (measured by a temperature sensor) with an exaggerated bounding pulse in the leg(s) (measured by a piezoelectric sensor). This may greatly decrease the risk that a false alarm will be generated. In further contrast to prior art monitoring systems, in which alerts may be produced unreliably if the animal is in a specific environment, or in a certain state or context that may mask a healthy condition, the system and method of the present invention may combine certain sensor data with data identifying the specific environment or state of the animal, thereby avoiding a false alarm. For example, if the multiple sensors merely detect bioparameters including pulse rate, then a false alarm for a particular medical condition like acute or chronic pain may result, whereas the system of the present invention may combine that pulse rate data with movement and position data identifying whether the animal is excited or running, which could explain the increased pulse rate without illness. Likewise, other states, environments or contexts such as exercising in hot weather could create a false alarm for a medical condition whose suspected diagnosis is grounded in part on increased temperature. Similarly, sleeping could create a false alarm for a medical condition whose suspected diagnosis is grounded in part on low pulse rate. Accordingly, the one or more processors of the system of the present invention may combine the pulse rate and other sensor data together with data identifying the state or environment of the animal, in order to reach a much more reliable determination as to whether the particular medical condition is suspected and thereby reduce the chance of a false suspected diagnosis and alarm. In still further contrast to prior art monitoring systems, the system of the present invention may include one or more remote and local processors. For example, a local processor on a band or collar may relay data to a remote processor in a server computer located in cyberspace. The system of the present invention, in contrast to the prior art, may interpret the interdependence of the vital sign measurements made by the sensor array to arrive at a suspicion of a medical diagnosis that may be relayed to a veterinarian, the animal's owner and/or to the authorities. Alternatively, the system of the present invention may relay a fitness of the horse or other animal to a trainer who may adjust a training regimen of the horse, for example a race horse. In still further contrast to the prior art, the band or collar may have two-way communication so that a veterinarian can instruct the band or collar to measure a particular vital sign remotely. In still further contrast to the prior art animal collars, in which signal to noise ratio precludes remote telecommunication reception of vital sign parameters, the band or collar of the present invention may include a layer of elastic that improves the signal to noise ratio by absorbing friction from constant movement of the horse or animals's head. In contrast to the prior art band or collars, the band or collar of the present invention may also have a GPS and communications system for alerting remote personnel so that if the animal is out of a designated area, or if a captive animal in a zoo escapes its enclosure, an immediate alarm can be sounded and an alert transmitted to designated authorities and veterinarians. In further contrast to prior art methods and appartuses of monitoring the animals' vital signs, which may interfere with the animal's behavior or cause irritation, for example because the band or collar has to be too tight, or which may not be sufficiently effective in capturing the low frequency sounds made by the animal, due to the fur of the animal around the neck or leg of the animal, the collar and method of the present invention may provide an effective method of monitoring the vital signs and diagnosing the health condition of the animal accurately without adversely affecting the behavior or comfort of the animal. Despite its accuracy, the band or collar may only need to gently touch the animal's leg(s) or neck, at several points around the neck. In still further contrast to the prior art animal bands or collars, in which signal to noise ratio precludes remote telecommunication reception of vital sign parameters, the band or collar of the present invention may include a layer of elastic material that may improve the sensitivity, gain and signal to noise ratio by absorbing friction from constant movement of the animal's head. In still further contrast to the prior art collars, which may not be accurate in capturing the low frequency sounds made by the animal, the band or collar and method of the present invention may utilize an acoustically enhanced band or collar for positioning on a neck or at least one of the leg's of the animal may have a working surface and a rear surface, at least one and preferably at least two acoustic concentrators projecting toward the leg(s) or neck of the horse from the working surface on one side of the at least one sensor element and at least one and preferably at least two acoustic balancers projecting from the rear surface on the other side of the at least one sensor. An acoustic balancer may be positioned at least partly behind a corresponding acoustic concentrator, and in some preferred embodiments the positioning maybe such that most or all of the acoustic concentrators have an acoustic balancer at least partly behind it on opposite sides of the band. In further contrast to prior art bands or collars, in which the structure of the device does not optimize capturing the low frequency sound by creating a balanced acoustic signal that is readable and able to be parsed, the band or collar of the present invention may have enhanced ability to convert low mechanical pulses to electrical signals of reasonable magnitude for transmission to the processor and thereby capture the low frequency sound effectively as a balanced acoustic signal that is readable and able to be parsed. Furthermore, in contrast to the prior art, the acoustic enhancers (concentrators and balancers) of the band or collar of the present invention may perform this function while simultaneously reducing total noise by reducing relative movement between the band or collar and the animal's head when the animal's head or leg(s) moves. The acoustic concentrators in the form of bumps may prevent occasional rotation of the band/collar relative to the leg(s) or neck of the animal. Instead, the band or collar of the present invention may move with the animal's leg(s) or head when the animal turns its head or moves its leg(s) up or down, due to the inward facing acoustic concentrators. Keeping the band/collar at the desired place may be critical both for the accuracy of the acoustic sensor and for the accuracy of the acceleration/position sensor that may be on the band/collar. As a result of the acoustic concentrators and acoustic balancers in a preferred embodiment of the system 11 of the present invention, the signal to noise ratio expressed using the logarithmic decibel scale may be at least 20 dB and in some preferred embodiments between 20 dB and 40 dB. The typical pulse amplitude is between 300 and 500 mV, as defined, by the gain setting of the amplifier (not shown). By having a better signal to noise ratio, in contrast of the prior art, the system of the present invention may be better able to provide reliable data that passes a quality assurance test, and hence may be able to provide a reliable suspicion of a medical condition with fewer sensors.

The principles and operation of a system, apparatus and method for an animal band or collar for health & vital signs monitoring, alert and diagnosis may be better understood with reference to the drawings and the accompanying description.

As seen from FIGS. 1-13, especially FIGS. 1-3B, a preferred embodiment of a system of the present invention may be a system 11 for monitoring vital signs of an animal. System 11 may comprise a band 20, such as an annular band 20, which may be in the form of a collar. Band 20 may have a working surface 20a configured to wrap around a portion of an animal. Although band 20 may be annular, being “wrapped around” the neck and/or at least one leg of the horse or other animal does not necessarily mean that the band stretches across the entire circumference of the leg or neck, although in one preferred embodiment band 20 does wrap around the entire neck and/or around the entire leg(s) of the animal. The band may comprise a layer of an elastic material. Band 20 may also have a rear surface 20b facing an opposite direction to the working surface 20a. If the band is around a neck of the animal, the band may also be called a collar. The term “band/collar” refers to a band that may be on the neck or may instead be on a leg(s) of the animal.

In one preferred embodiment, system 11 may include a sensor array comprising at least one sensor element situated along a circumference of the band, the at least one sensor element including at least one of (i) an accelerometer configured to measure at least one accelerometer-measured bioparameter of the animal from among: resting patterns, activity patterns, movement patterns, position patterns, lameness, kicking, stomping, lifting leg, pawing and (ii) a non-accelerometer sensor configured to measure at least one of the following non-accelerometer-measured bioparameters of the animal: temperature, pulse rate, respiration rate.

The sensor array may comprise at least two sensor elements situated along a circumference of the band 20, the at least two sensor elements including an accelerometer and a non-accelerometer sensor. The at least two sensor elements may comprise at least three or at least four or at least five or at least six or at least seven (or more) sensor elements distributed at different points along the circumference of the band.

System 11 may also comprise one or more remote (40A) or local processors 40 configured to receive (a) sensor output data from the sensor array concerning the measured bioparameter(s) and (b) reference data concerning the measured bioparameters of the animal or of a population of the animal.

The one or more remote or local processors may include one or more local processors 40 and/or one or more remote processor 40A.

The one or more local or remote processors 40, 40A may be configured to determine whether a specific medical condition is suspected by utilizing at least one of the following: (i) scoring at least two bioparameters relative to the reference data and comparing a cumulative score of all scored bioparameters to a threshold cumulative score or to a threshold cumulative range; or (ii) identifying an abnormal pattern in at least one bioparameter from among the accelerometer-measured bioparameters and the non-accelerometer-measured bioparameters (or if there only at least one sensor in the sensor array, then (ii) identifying an abnormal pattern in at least one bioparameter from among the at least one of (I) the accelerometer-measured bioparameters and (II) the non-accelerometer-measured bioparameters).

The one or more remote or local processors may be configured to send an alert if at least one specific medical condition is suspected. Accordingly, as seen in FIG. 11, in some preferred embodiments of the system of the present invention, the system may further comprise a two-way communication device, which may be attached to band 20, for communicating the suspicion in a form of an alert to a remotely stationed veterinarian or other user.

Note that if the one or more local or remote processors of system 11 consist solely of local processors 40 (i.e. processors situated in or on or at band/collar 20), then the system 11 can also be described as an apparatus or device 20, which apparatus or device may be described as a band or collar 20 having various components. On the other hand, if the system 11 includes at least one remote processor 40A, or even one remote device such as a commutation device, the system 11 is not an apparatus but includes an apparatus such as a band 20 or collar 20.

In system 11, the one or more processors 40, 40A may be configured to combine the identifying of the abnormal pattern in the at least one bioparameter with identifying abnormal patterns in at least one other bioparameter. For example, the identifying of the abnormal pattern in the at least one bioparameter involves identifying said abnormal patterns in at least one accelerometer-measured bioparameter and identifying abnormal patterns in at least one non-accelerometer-measured bioparameter.

If, for example, the specific medical condition is colic and the abnormal pattern in the at least one accelerometer-measured bioparameters is an abnormal pattern of movement comprising at least one of pawing; kicking; stomping; lifting leg; repeatedly lying down and rising; rolling or thrashing; change in activity level (i.e. lethargy, pacing or a constant shifting of weight when standing); stretching or abnormal posturing; or dorsal recumbency in foals, the abnormal pattern of movement may be sensed by the accelerometer.

In preferred embodiments, the sensor output data from the sensor array concerning the bioparameters may be tested to see if it passes a quality assurance test. The quality assurance test may be based on a threshold level of signal to noise ratio. Accordingly, the at least one acoustic concentrator and at least one balancer that are utilized in a preferred embodiment may greatly improve the signal to noise ratio and allow the data to pass the quality assurance test. In certain other preferred embodiments, the quality assurance test may be based on a pattern recognition. In still other preferred embodiments, the bioparameters are to see if they pass a quality assurance test, wherein the quality assurance test is based on whether a quantity of data points of the data is sufficiently high.

The following is an example of certain logic used in combining data from different sensors (data fusion) by the one or more local or remote processors in accordance with certain preferred embodiments. For the medical condition of colic, a non-accelerometer sensor senses pulse data (“A”) and detects elevated pose rate. This detection is considered a necessary component for detecting this condition in accordance with this preferred embodiment. At the same time, the accelerometer sensor senses activity data (“B”) to confirm that the elevated pulse data pattern must happen while the animal is resting. According to this logic, pulse data+activity (A+B) parameters are necessary components. (A+B) parameters may even be considered as sufficient components, if the data relating to them is considered of good enough quality (high confidence), i.e., if the incoming data receives a passing score when subjected to one or more quality assurance tests.

If, on the other hand, the activity and position incoming data (A+B) is merely of medium quality or of borderline confidence, and hence inconclusive, then supplemental data from the following other sensors that may sense other parameters may support a suspicion of colic. The supplemental data may be position data that shows for example the horse rolling on its back. This is a strong supportive component for a suspicion of colic. The supplemental data may be respiration data that shows increased respiration rate at rest—this is a supportive component for a suspicion of colitis. The supplemental data may be data concerning sounds from an acoustic accelerometer, i.e. whinnying, grunting. This is a supportive component for a suspicion of colic. In sum, in this preferred embodiment, A+B at a level of good score on a quality assurance test would yield an alert. Furthermore, A+B at a level of a medium score on a quality assurance test plus one or more supplemental data would yield an alert.

In one preferred embodiment, for the medical condition of colic, laminitis and/or lameness, each parameter and basic/background attributes may be assigned a pre-determined score. No one parameter is necessary but rather a sufficient accumulation of supporting parameters. The scores of all parameters are summed and if at any time the cumulative score passes a threshold score, then an alert is generated.

In one preferred embodiment, for the specific medical condition of colic the abnormal pattern in the at least one accelerometer-measured bioparameters is an abnormal pattern of movement comprising at least one of pawing; kicking; repeatedly lying down and rising; rolling or thrashing; change in activity level including lethargy or pacing or a constant shifting of weight when standing; stretching or abnormal posturing; or dorsal recumbency in foals sensed by the accelerometer. In this case, the non-accelerometer-measured bioparameters may be at least one of elevated body temperature measured by a temperature sensor, change in the degree of gut sounds measured by a sound sensor, groaning measured by a sound sensor and loss of appetite measured by a proximity sensor.

In one preferred embodiment, the one or more remote or local processors may be configured to determine a suspicion of all (a) colic, (b) laminitis and (c) lameness based on at least one of movement, pulse, temperature and respiration, wherein pulse means pulse rate or pulse rhythm. In other preferred embodiments, the one or more remote or local processors may be configured to determine a suspicion of any one or two of (a) colic, (b) laminitis and (c) lameness based also on at least one of movement, pulse, temperature and respiration, wherein pulse means pulse rate or pulse rhythm.

In one preferred embodiment, wherein the at least one specific medical condition is laminitis, the one or more remote or local processors are configured to base a suspicion of laminitis at least in part on scoring at least two bioparameters from among (i) loss of appetite, (ii) decreased activity (iii) abnormal standing posture, (iv) walking with a slow, crouching, short-striding gait, (v) elevated skin temperature above the hoof area on the lower leg (i.e. up to 20 centimeters from the hoof), (vi) an exaggerated and bounding pulse in the leg, (vii) severe pain (viii) increased pulse rate (ix) increased respiratory rate, and (x) an increased amount of time lying down, and comparing a cumulative score of all scored bioparameters to a threshold cumulative score or to a threshold cumulative range.

In one preferred embodiment, wherein the at least one specific medical condition is lameness, the one or more remote or local processors are configured to base a suspicion of lameness at least in part on scoring the at least two bioparameters from among (i) shifting weight, (ii) abnormal positions, (iii) head bobbing, (iv) decreased activity, (v) pain, (vi) bounding pulses, wherein “(i)” through “(iv)” are measured by the accelerometer and “(v)” through “(vi)” are measured by a piezoelectric sensor.

In the present invention in general, reference to configuring the one or more local or remote processors refers to programming the one or more local or remote processors with software or programmable data instructions. The software or programmable data instructions referred to in the present invention is normally stored on a non-transitory computer-readable medium or memory storage and is executed by the one or more local or remote processors.

In general, the one or more local or remote processors may be configured to determine whether each of the at least two bioparameters exceeds a threshold level or range. Alternatively, the one or more remote or local processors may be configured to determine whether a new parameter that is a function of a combination of each of the at least two relevant bioparameters, and may be configured to determine if the new parameter exceeds a threshold level or range.

In general, in order to combine data received from a sensor to include information about environments or states of the animal, the sensor array may be configured to measure at least one of the following characteristics of the animal for output to the one or more remote or local processors: skin temperature on the leg above the hoof or on the neck, heart rate, respiratory rate, the degree of gut sounds and changes in said degree, movement patterns including pawing, kicking, repeatedly lying down and rising, rolling, thrashing, changes in activity level (i.e. lethargy, pacing or a constant shifting of weight when standing), stretching, abnormal posturing, groaning, loss of appetite, dorsal recumbency in foals, decreased activity, abnormal standing posture, a slow, crouching, short-striding gait when forced to walk, an exaggerated or bounding pulse in the leg(s), severe pain, increased pulse rate, increased respiratory rate, increased amount of time lying down, shifting weight, abnormal positions, head bobbing, pain or bounding pulses.

For example, for the specific medical condition of colic, the one or more remote or local processors may be configured to base a suspicion of colic at least in part on scoring at least two bioparameters from among the following clinical signs, where the clinical sign(s) may be detected by the sensor identified in the parenthetical expression following that clinical sign(s): elevated body temperature as determined by skin temperature on one or more legs above the hoof or else on the neck (temperature sensor); elevated heart rate (piezoelectric sensor); elevated respiratory rate (piezoelectric sensor); change in the degree of gut sounds (sound or acoustic sensor); movement patterns including pawing (accelerometer); kicking (accelerometer); repeatedly lying down and rising which may become violent when the colic is severe (accelerometer); rolling, thrashing (accelerometer); changes in activity level: lethargy, pacing or a constant shifting of weight when standing (accelerometer), stretching, abnormal posturing (accelerometer); groaning (sound sensor); loss of appetite (accelerometer, proximity sensors); and dorsal recumbency in foals (accelerometer).

Likewise, for the specific medical condition, of laminitis, the one or more remote or local processors are configured to base a suspicion of laminitis at least in part on scoring at least two bioparameters from among (i) loss of appetite (sound sensors); (ii) decreases activity (acceleromter); (iii) abnormal standing posture (accelerometer); (iv) if forced to walk, the horse shows a slow crouching, short-striding gait (accelerometer); (v) skin temperature near the hoof are may be elevated (temperature sensor); (vi) an exaggerated or bounding pulse in the leg (piezoelectric sensor); (vii) severe pain (viii) increased pulse rate (piezoelectric sensor); (ix) increased respiratory rate (piezoelectric sensor); and (x) increased amount of time lying down (accelerometer), comparing a cumulative score of all scored bioparameters to a threshold cumulative score or to a threshold cumulative range; and measuring a persistence over time of either the cumulative score or the abnormal pattern.

If the accelerometer is an acoustic accelerometer configured to measure is sounds, it may be configured to measure a presence of at least one or at least two horse sounds (or sounds of other members of the equidae family), or in other preferred embodiments, at least three horse animal sounds, or at least four horse sounds (or in other preferred embodiments at least five or at least six or seven or eight or at least nine or ten or eleven) among groaning, whinnying, grunting.

If the animal is a horse, the system may be configured to determine a suspicion of at least one or at least two or at least three, or at least four of the following specific medical conditions of horse: colic, laminitis, lameness, fitness of race horses.

In certain preferred embodiment of the system of the present invention, instead of both accelerometers and other sensors, the sensors of system 11 may be limited to one or more accelerometers situated along a circumference of the band. The one or more accelerometers may include acoustic accelerometers and non-acoustic accelerometers. The one or more accelerometers may be configured to measure at least one bioparameter of the animal from among: resting patterns, activity patterns, movement patterns, position patterns, lameness, kicking, stomping, lifting leg, pawing. If the sensors are limited to one or more accelerometers, the one or more remote or local processors may be configured to receive (a) sensor output data from the one or more accelerometers concerning the measured bioparameters and (b) reference data concerning the measured bioparameters of the animal or of a population of the animal, the one or more remote or local processors configured to determine whether a specific medical condition is suspected by at least one of the following: (i) scoring at least two bioparameters and comparing a cumulative score of all scored bioparameters to a threshold cumulative score or to a threshold cumulative range; or (ii) identifying an abnormal pattern in at least one accelerometer-measured bioparameter.

As seen from FIGS. 1-3B, one version of the system or method of the present invention may utilize an acoustically enhanced collar 20 for monitoring vital signs of an animal of the Equidae family. For example, a collar 20 may include a band 20 that may comprise a layer of an elastic material, for example polyurethane. The elastic material may include any kind of plastic or other flexible material, although in a preferred embodiment, elastic material may comprise polyurethane. The band 20 may be configured for cushioning repetitive instances of friction against the band or collar 20 from movement of the head or leg of the animal.

Band 20 may is located on the neck of the animal and/or (as seen from FIG. 13A) on at least one leg of the animal. For any leg that the band 20 is placed on, band 20 may be positioned adjacent the hoof. Preferably, the band is situated on the leg above and adjacent to the hoof. In one preferred embodiment, band 20 is within 2-5 centimeters (or 2-10 centimeters in other preferred embodiments) of the hoof on the leg. In other preferred embodiments, the band 20 is above the hoof and within 20 centimeters of the hoof.

In order to protect band 20 and keep it in place, in certain preferred embodiments, band 20 is closed inside a boot that a horse wears. In other preferred embodiments, the band 20 is wrapped with an elastic wrapping or bandage that the horse wears.

As seen from FIGS. 1-3B, band 20 may have a working surface 20a that may be configured to wrap around a portion of an animal. As shown in FIG. 11 and FIG. 12, the portion of the animal may be one or more legs 17 of the animal 18. Band 20 may have a rear surface 20b that may face an opposite direction from working surface 20a. For example, working surface 20a may face the animal's neck (or leg(s)) and rear surface 20b may face a viewer looking directly at the band 20 on the neck (or leg(s)) of the animal. Band 20 (and collar 20) may be approximately two inches wide and may cover an entire circumference of the neck of the animal (or alternatively most or a portion of this circumference). There may be sensors 30, for example four or more sensor elements 30 at different points of the band 20, preferably at different points along a length or circumference of band 20. There may be other numbers of sensor elements, such as one, two, three, five, six, seven, eight, nine or ten and more.

As seen from FIGS. 1-3B, the band 20 may have at least one acoustic concentrator projecting toward a body portion of the animal from the working surface at one or more regions along the circumference; and may have at least one acoustic balancer projecting from the rear surface at the one or more regions along the circumference. The at least one acoustic balancer may be situated at a region along the circumference at least partly behind the at least one acoustic concentrators.

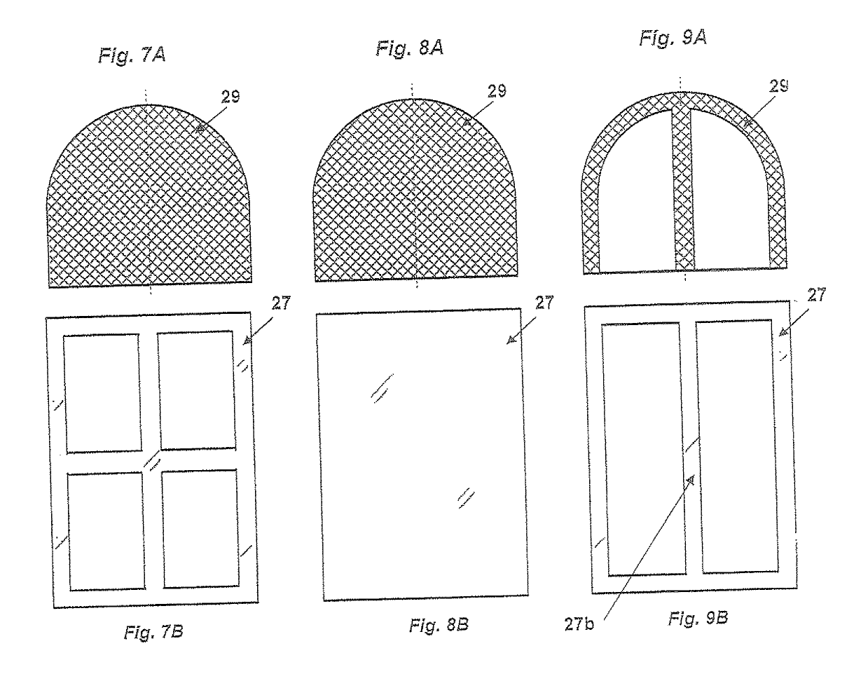

Each acoustic concentrator 22 may have a concentrator base end 23, which refers to the base of the acoustic concentrator adjacent the working surface 20a, (see FIGS. 4B, 5B, 6B). Each acoustic concentrator 22 may have a concentrator top end 25 (see FIGS. 4A, 5A, 6A), the end projecting furthest from the band 20 and closest to the animal's neck and/or at least one leg. Likewise, each acoustic balancer 24 may have a balancer base end 27 (see FIGS. 7B, 8B, 9B) and a balancer top end 29 (see FIGS. 7A, 8A, 9A).

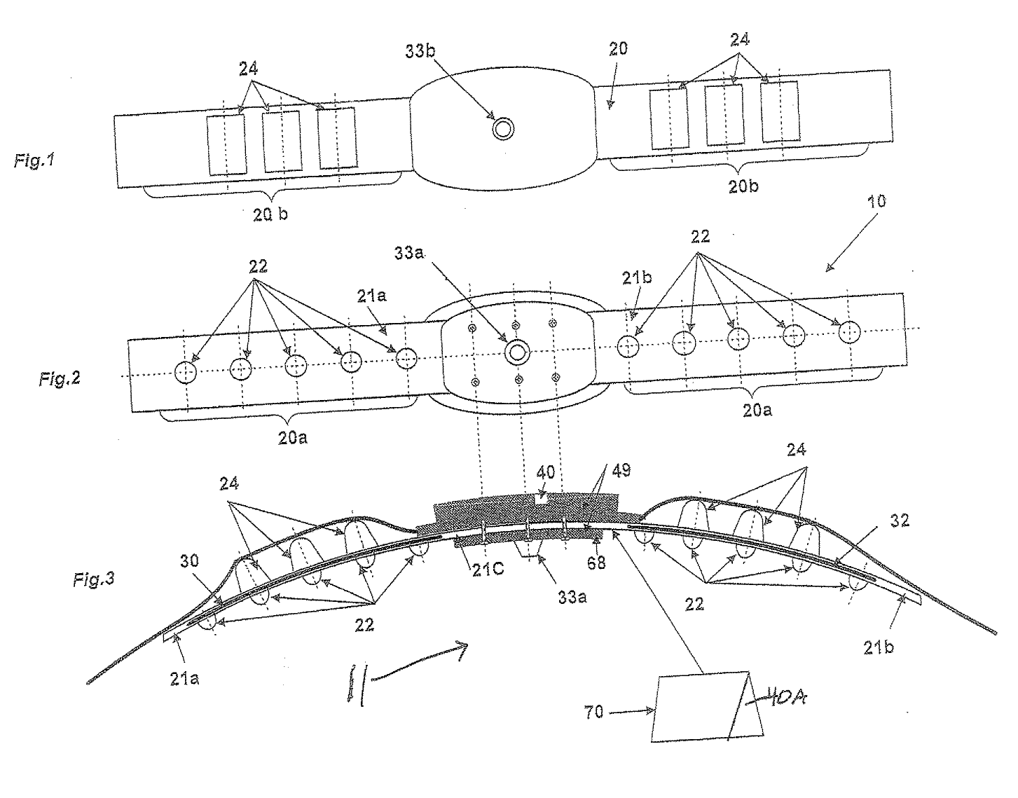

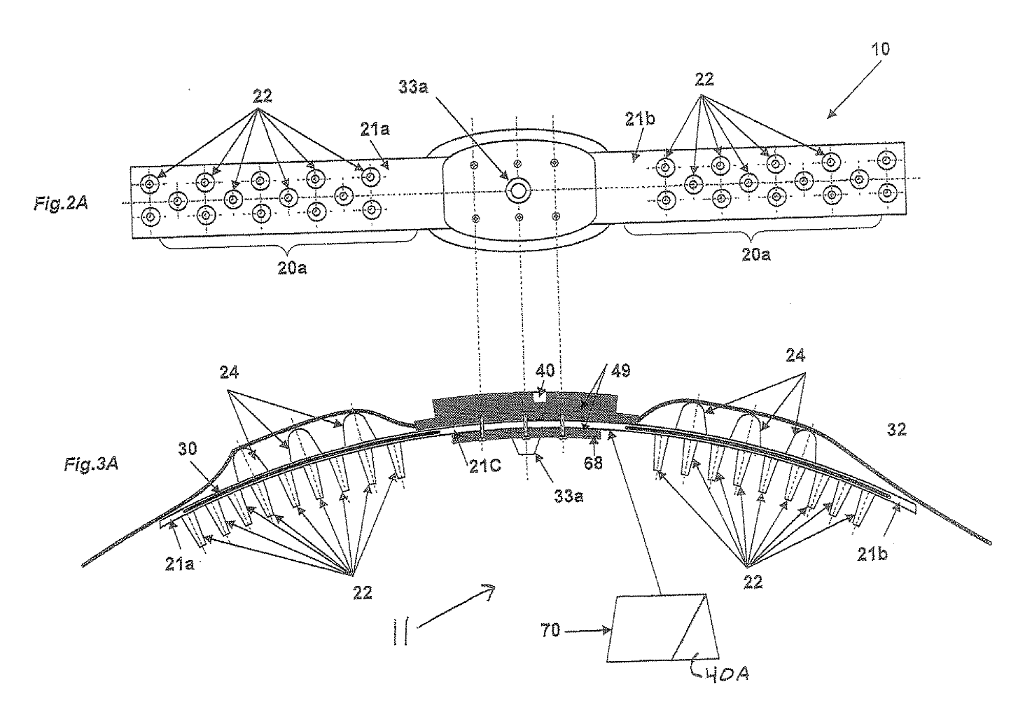

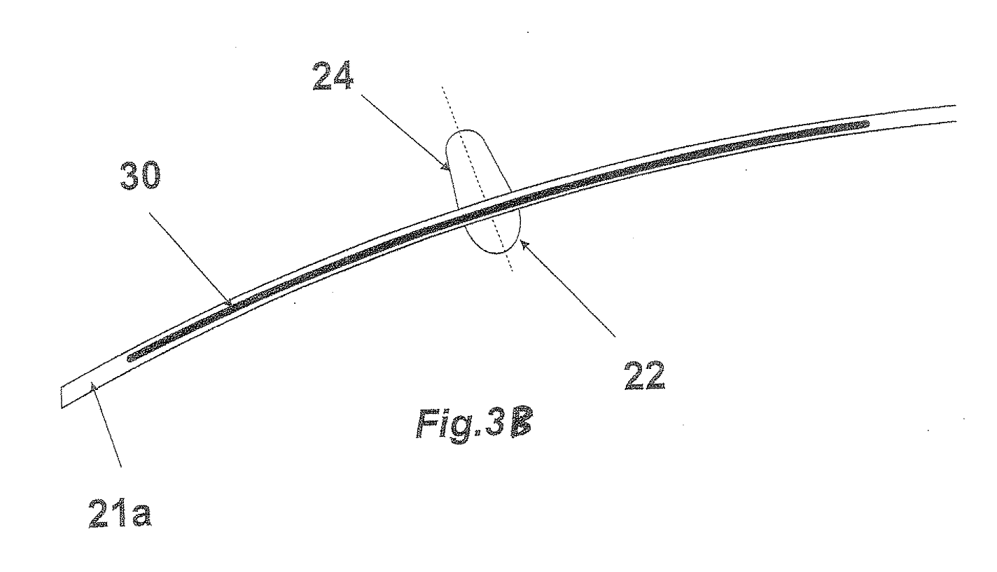

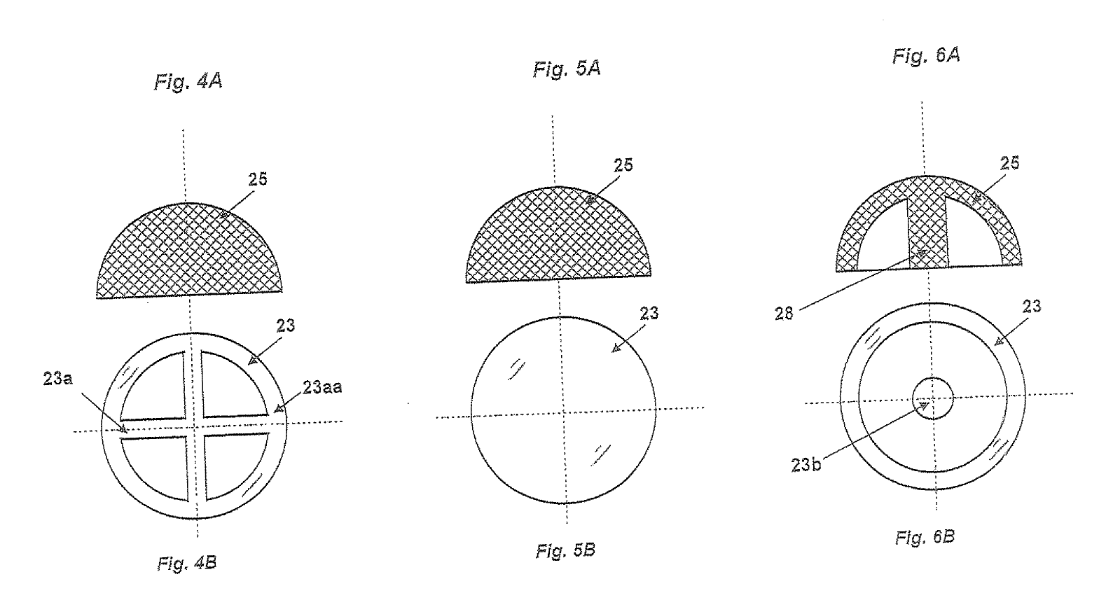

In a preferred embodiment, in which we consider an acoustic concentrator 22 and an acoustic balancer 24 to constitute a “pair” as shown for example in FIG. 3, there are at least two pairs of acoustic concentrators 22 and acoustic balancers 24 for each sensor element 30, 32. The at least one sensor element 30 (on the left side of the band 20) and the at least one sensor element 32 on the right side of band 20 may each comprise a piezoelectric material which in a preferred embodiment may be embedded inside band 20. One preferred conical shape of the acoustic concentrators 22 is shown in FIG. 3A. In FIG. 2A, the concentric circles depicting the bottom view of acoustic concentrators 22 show that the concentrators 22 are conical and may have a circular base.

If there are two sensors elements, then the sensors 30 may be connected in parallel electrically (the at least one sensor element 30 may comprise two physically separated sensors connected electrically). One can also define the two sensor elements 30 as one distributed sensor element. Positioning two sensors 30 on the two sides of the neck of the animal may provide a guaranteed contact with the body regardless of movement or position.

In one preferred embodiment used in the system or method of the present invention, band 20 may have at least two acoustic concentrators 22, projecting toward a neck or other portion of the animal from the working surface 20a at the one or more regions along the circumference of the band 20 that the at least one sensor element may be situated in. Likewise, band 20 may have at least two acoustic balancers 24 projecting from the rear surface 20b at the one or more regions along the circumference of band 20 that the at least one sensor element may be situated in. As seen from FIG. 3, the at least two acoustic balancers 24 may be situated at a region along the circumference of the band 20 that is at least partly behind the at least two acoustic concentrators the at least one sensor 30, the at least two acoustic balancers situated opposite the at least two acoustic concentrators. Preferably, the at least two acoustic concentrators and the at least two acoustic balancers are situated so that one acoustic concentrator is directly opposite one corresponding acoustic balancer, as shown in FIG. 3. In some preferred embodiments, as shown in FIG. 3, the base end 23 of at least one acoustic balancer 24 is at least as wide as the base end 27 of the acoustic concentrator 22 that it is behind. More preferably, the one acoustic concentrator that is opposite its corresponding one acoustic balancer is of the same or similar shape as the corresponding one acoustic balancer. This may mean that a pair comprising one acoustic concentrator 22 and one acoustic balancer 24 at least partly behind it have the same overall shape, but in other preferred embodiments, it may mean that they have the same width, or that they have the same cross-section or the same shape at their base end 23 (the end facing the band 20) or the same width at their base end, or both have perpendicular diameters at their base ends or other structural similarities.

The acoustic concentrator 22 may touch the skin of the animal and absorb the noise from friction while conducting the signal and may penetrate the fur on the neck of the animal (or on the leg(s)) without causing the animal discomfort. This may be arranged by configuring the height of the projection (its length from the sensor element 30 substantially perpendicularly toward the neck of the animal) and thereby controlling how far the acoustic concentrator projects toward the direction of the neck of the animal. The comfort of the animal may be verified by testing the band on various horses or other animals of the Equidae family (i.e. zebras, donkeys).

In one preferred embodiment used in the system or method of the present invention, as shown in FIGS. 1-3A, band 20 may include at least one sensor element 30 situated at one or more regions along a circumference of the band 20 and configured to measure at least one bioparameter relating to vital signs of the animal. The term “region” is not intended to indicate how much length the region has and the region can be as long as the band 20 or as narrow as a line, although as shown in FIG. 3 and FIG. 3A, it is preferably elongated along a circumference of the band 20. In a preferred embodiment, the at least one sensor element may be configured to measure at least one bioparameter from the following bioparameters: temperature, heart rate, respiration rate, movement. Preferably, the at least one sensor element is configured to measure at least two of the bioparameters, and more preferably at least three and most preferably all four of them.

The neck is a particularly suitable portion of the animal to monitor since it not only allows listening acoustically to two major blood vessels (arteries) but also monitoring a breathing pipe (the windpipe). It has also been found that the legs of a horse is a particularly suitable portion of the animal to monitor since large blood vessels are located in the back of the leg above the hoof, which allows capturing the pulse and respiration easily, and with a favorable signal to noise ratio.

As shown in FIGS. 1-2A, band 20 may have a first side 21a and a second side 21b along its circumference. These “sides” may be portions of the circumference of band 20, for example on each side of a centrally located sensor element 33 (FIG. 3) (for example a temperature sensor 33a for measuring body temperature as shown in FIG. 2 and a temperature sensor 33b for measuring ambient temperature as shown in FIG. 1) and these “sides” should not be confused with the “vertically” opposite sides (working surface and rear surface) of or in relation to a sensor element 30 that the acoustic concentrators 22 and acoustic balancers 24 may be on. The first side 21a along the circumference of the band 20 may be on the left side of the neck of the animal and the second side 21b along the circumference of the band 20 may be the right side of the neck of the animal or vice versa. This may allow a most preferable embodiment in which at least one sensor element is positioned on the first side of the neck and at least one sensor element is positioned on the second side of the neck. FIG. 3 also shows a central portion 21c of the circumference of band 20 that may be located between sides 21a, 21b.

At least one sensor element 30 may be situated at one or more regions along a circumference of the band 20, and the at least one sensor element 30 may be configured to measure at least one bioparameter from the following bioparameters: temperature, heart rate, respiration rate, movement. As shown in FIG. 1, FIG. 3 and FIG. 3A, band 20 may also include at least one acoustic concentrator 22 projecting as a bump toward the portion of the animal (for example the neck) from the working surface 20a. The at least one acoustic concentrator 22 may be situated at the one or more regions along the circumference and on a first vertical side (vertically speaking by reference to FIG. 3) of the at least one sensor element 30 that may be embedded in the band 20 (see FIG. 3). As also seen from FIGS. 2-3, at least one acoustic balancer 24 may be situated along the one or more regions of the circumference and may be projecting as a bump from the rear surface 20b of the band 20 on a second vertical side of the at least one sensor element. The position of the at least one acoustic balancer along the circumference of the band may be that it is at least partly behind, and preferably directly behind, the at least one acoustic concentrator 22.

As also shown in FIG. 3, a similar arrangement may exist on the other side 21b of the circumference of the band 20 with another at least one sensor 30 and another at least one acoustic concentrator 22 and another at least one acoustic balancer 24 similarly configured for example behind, and preferably directly behind, the at least one acoustic concentrator 22. This may allow monitoring both sides of the neck of the animal.

One or two or preferably all of the at least one acoustic concentrator 22 are wider at a concentrator base end 23 adjacent the working surface 20a than at a concentrator top end 25. For example, in FIG. 3A, the acoustic concentrators are conical. One or two or preferably all of the at least one acoustic balancers 24 are wider at a balancer base end 27 adjacent the rear surface 20b than at a balancer top end 29. In one preferred embodiment shown in. FIGS. 4A-6B, the at least one acoustic concentrator 22 and the at least one acoustic balancer 24 are substantially circular in at least one dimension. An acoustic concentrator 22 of the at least one acoustic concentrator may be substantially semispherical. An acoustic balancer of the least one acoustic balancer may be substantially semispherical and may be located at least partly behind, and preferably directly behind, the acoustic balancer of the at least one acoustic concentrator on the first side 21a that is substantially semispherical. In other embodiments, the least one acoustic concentrator may be substantially semi-cylindrical (not shown) and the at least one acoustic balancer may be substantially semi-cylindrical (not shown) and located at least partly behind and preferably directly behind the one of the at least one acoustic concentrators that is substantially semispherical.

The acoustic concentrators 22 may be designed to optimize the acoustic transmission of sound vibrations from the animal's neck to the sensor element 30 in the band 20. In order to accomplish their purpose, the acoustic concentrators 22 and acoustic balancers 24 of the collar 20 may vary in terms of their size and in terms of their shape. With regard to their shape, an important aspect of their shape is the configuration of the base end of the acoustic concentrator or balancer. The base end of the acoustic concentrator (concentrator base end) and the base end of the acoustic balancer (balancer base end) are each closer to the sensor element 30 than the respective top ends (concentrator top end and balancer top end).

As shown in FIGS. 3, 3A, 4A, 5A, 6A, one or two or preferably all of the at least two acoustic concentrators 22 may be wider at a concentrator base end 23 adjacent the working surface 20a than at a concentrator top end 25. Likewise, as shown in FIGS. 3, 7A, 8A, 9A, one or preferably each of the at least two acoustic balancers 24 may be wider at a balancer base end 27 adjacent the rear surface 20b than at a balancer top end 29. The term “projecting” refers to the fact that the acoustic concentrators 22 and balancers 24 may project beyond the surface of the band 20, which may be relatively flat other than the acoustic concentrators and balancers. Although, these projections have been referred to as “bumps”, the term “bump” is not intended as a limitation on the shape of the projections, although in many preferred embodiments, the “bumps” look like curved protrusions akin to a bump. In certain preferred embodiments, the acoustic concentrators 22 and acoustic balancers 24 are rounded and symmetrical.

In one preferred embodiment, there are at least four sensor elements 30. Each sensor element 30 may be a strip of two inches to six inches in length depending on the size of the collar 20. In a preferred embodiment, there are at least two acoustic concentrators for each sensor element. For example, in one preferred embodiment where the sensor element is two and three-quarters inches, there may be four acoustic concentrators for that sensor 30. Acoustic concentrators 22 and acoustic balancers 24 may be located at the opposite sides of the sensor, as shown in FIG. 1 and FIG. 3. The collar 20 contains two sensors located symmetrically at the left and right sides of the neck.

The acoustic concentrators and the acoustic balancers may be integrally formed with the band and may be made of the same material as the band. For example, the bumps (acoustic concentrators and acoustic balancers) may be formed at the same time that the band is formed. Preferably, each of the at least two acoustic concentrators 22 are shaped like a bump, for example like a bump that diminishes in diameter from the concentrator base end to the concentrator top end. Likewise, in a preferred embodiment, each of the acoustic balancers is shaped like a bump, for example like a bump that diminishes in diameter from the balancer base end to the balancer top end. The bumps, in a preferred embodiment, may be mostly hollow except for particular structural elements in particular shapes that may fill the void of the hollow.

The acoustic concentrators 22 (or one particular acoustic concentrator of the at least one acoustic concentrator) and the acoustic balancers 24 (or one particular acoustic balancer of the at least one acoustic balancer) may be mathematically elliptical, for example substantially circular, in at least one dimension. Preferably, they may be substantially circular in two dimensions. As shown in FIGS. 2, 3, 4B, 5B, 6B, at least one, and preferably at least two, of the acoustic concentrators are substantially semispherical. Preferably, the shape of an acoustic balancer 24 mirrors the shape of the acoustic concentrators 22 that the balancer 24 faces on the opposite side of the band 20. Accordingly, preferably, at least one, and preferably at least two, of the at least two acoustic balancers 24 are substantially semispherical. In a different preferred embodiment (not shown), at least one of the at least two acoustic concentrators 22 is substantially semi-cylindrical and at least one of the at least two acoustic balancers 24 may also be substantially semi-cylindrical in this embodiment.

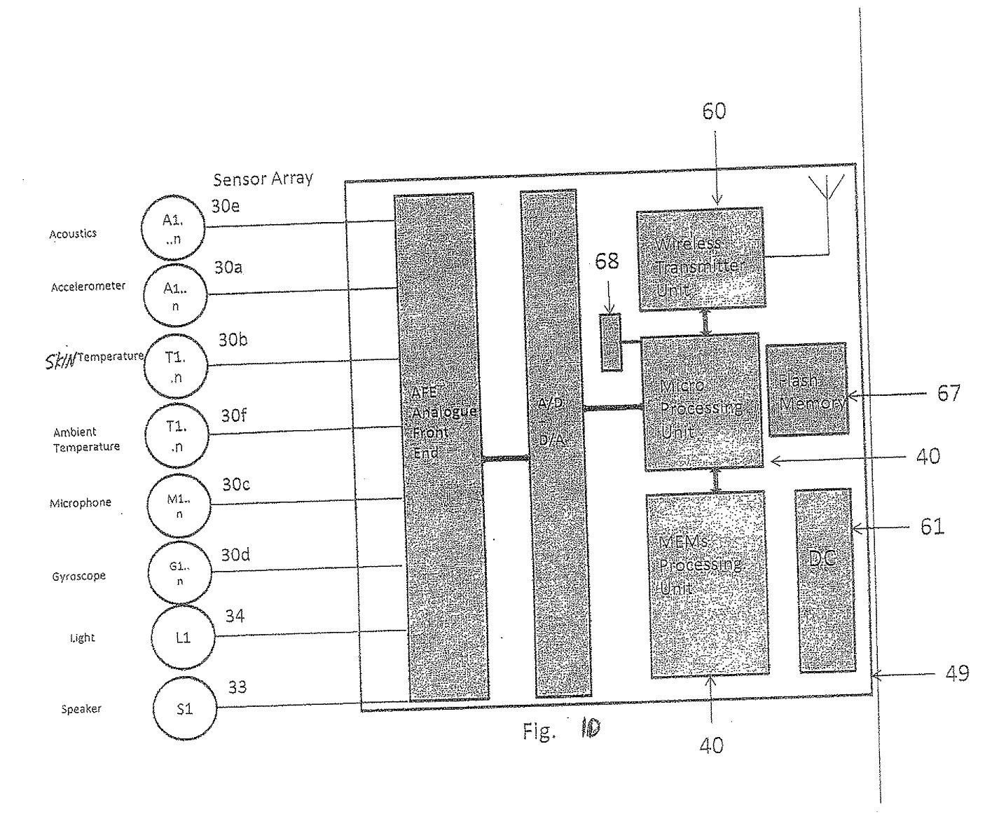

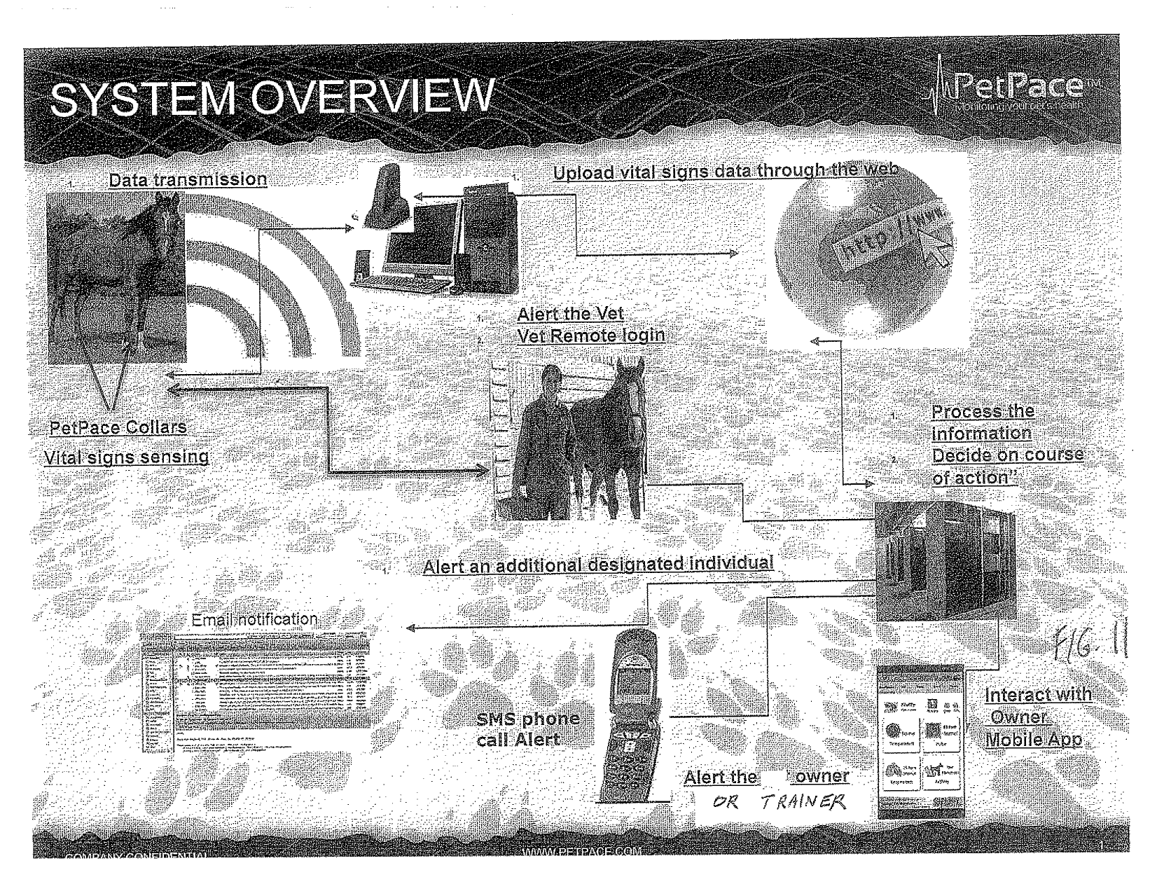

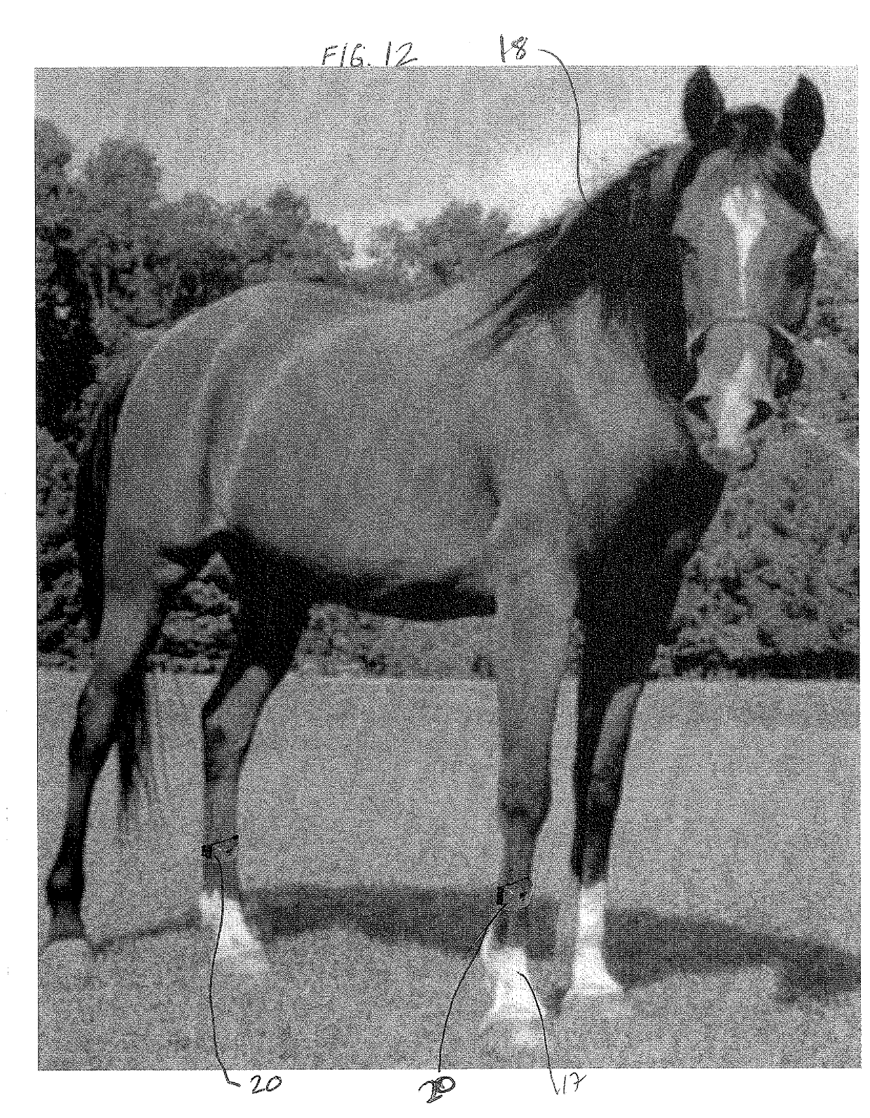

As shown in FIG. 6A-6B, each concentrator base end of the at least one or the at least two acoustic concentrators 22 may comprise a central dot 23b connected to the concentrator top end 25 along a linear axis 28. As shown in FIGS. 9A-9B, each balancer base end 27 of the at least one or the at least two acoustic balancers 24 may comprise a central dot 23b connected to a balancer top end 29 along a linear axle 28. In this embodiment, as shown in FIGS. 6A-6B and 9A-9B each acoustic concentrator 22 and each acoustic balancer 24 may be hollow except for the central dot and linear axle.

In one preferred embodiment of the concentrator base end 23 and the balancer base end 27, shown respectively in FIG. 4B and FIG. 7B, the X-shape 23a or perpendicular diameters may appear in a closed curve or a substantially closed curve. The “closed curve” may be a circular perimeter. As shown in FIG. 4B and FIG. 7B, the circular perimeter 23aa of the X-shape may be thicker than a thin outer perimeter and could in some preferred embodiments be thick enough to occupy 20% to 20% of the diameter/width of the concentrator base end 23 or of the balancer base end 29 (and in certain other preferred embodiments 5% to 20% or 15% to 30% or 3% to 6% or about 3% or about 5%, or about 20%, or about 15%, or about 20%, or about 25% or about 30% or other numbers depending on the embodiment of the diameter/width of the concentrator base end 23 or of the balancer base end 29). This proportion presupposes that the thickness of the perimeter is included in the calculation only once (not twice due to the two parts of the perimeter appearing 180 degrees apart from on another). As shown in FIG. 4A and FIG. 7A, the cross-section of the acoustic concentrators 22 and the acoustic balancers 24 may in the shape of an “X”.

In certain other preferred embodiments, the acoustic concentrators and acoustic balancers are solid, as shown in FIG. 5B and FIG. 8B. In one preferred embodiment shown in FIGS. 2A and 3A, acoustic concentrators 22 are shaped like a cone.

Depending on the shape, the bumps comprising the acoustic concentrators and acoustic balancers may have a diameter of between 5 and 7 millimeters. Depending on the shape, the acoustic concentrators and acoustic balancers may also have a height of between 5 and 7 millimeters.

As a result of the acoustic concentrators and acoustic balancers in a preferred embodiment of the system 11 of the present invention, the signal to noise ratio expressed using the logarithmic decibel scale may be at least 20 dB and in some preferred embodiments between 20 dB and 40 dB. The typical pulse amplitude is between 300 and 500 mV, as defined by the gain setting of the amplifier (not shown).

In general, sensor elements 30 may be at least one sensor element 30 designed or configured to measure at least one bioparameter from among temperature, heart rate, respiration rate and movement. Alternatively, the sensor element may be for measuring a different vital sign. There could be more sensor elements and more bioparameters. For example, the at least one sensor element 30 may comprise at least two sensor elements 30 that may be configured or designed to measure at least two bioparameters from among temperature, heart rate, respiration and movement. Alternatively, the at least two sensor elements 30 may be for measuring at least two bioparameters from among temperature, heart rate, respiration rate and movement (or alternatively other vital signs). One sensor element may measure multiple bioparameters, for example, in the case of an acoustic sensor that measures respiration rate and heart rate. The at least two sensor elements may comprise four or more sensor elements designed to measure four or more bioparameters or specifically those four: temperature, heart rate, respiration rate and movement. In some preferred embodiments, the array of sensor elements 30 are designed to measure one or two bioparameters (in other preferred embodiments three or four) from the following bioparameters: temperature, heart rate, respiration rate, movement (for example horizontal and vertical movement) and positions.

The sensor elements 30 may be designed or configured to measure at least two different vital sign bioparameters as well as to measure certain bioparameters, such as movement, that may be useful in understanding the horse's vital signs when combined with other vital sign bioparameters. Each of the various sensor elements 30 on the band 20 may be designed for measuring a different vital sign parameter or in some cases there may be more than one sensor element measuring a particular vital sign bioparameter or more than one vital sign measured by a particular sensor element 30.

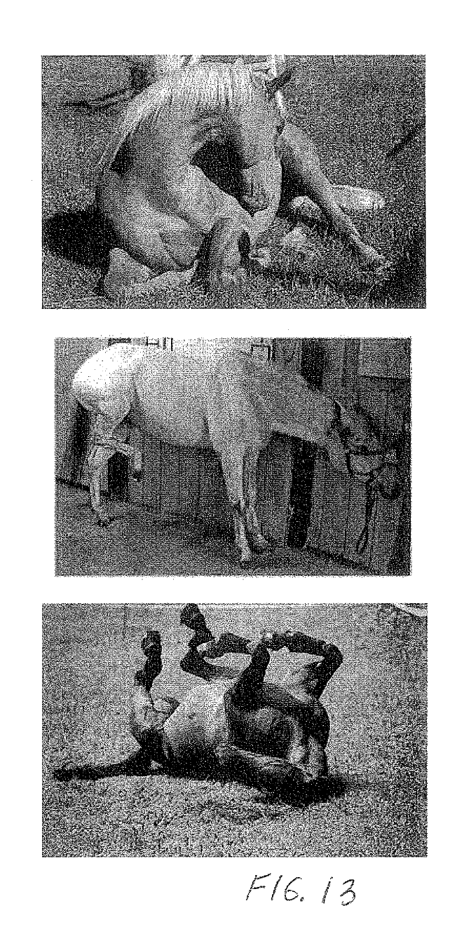

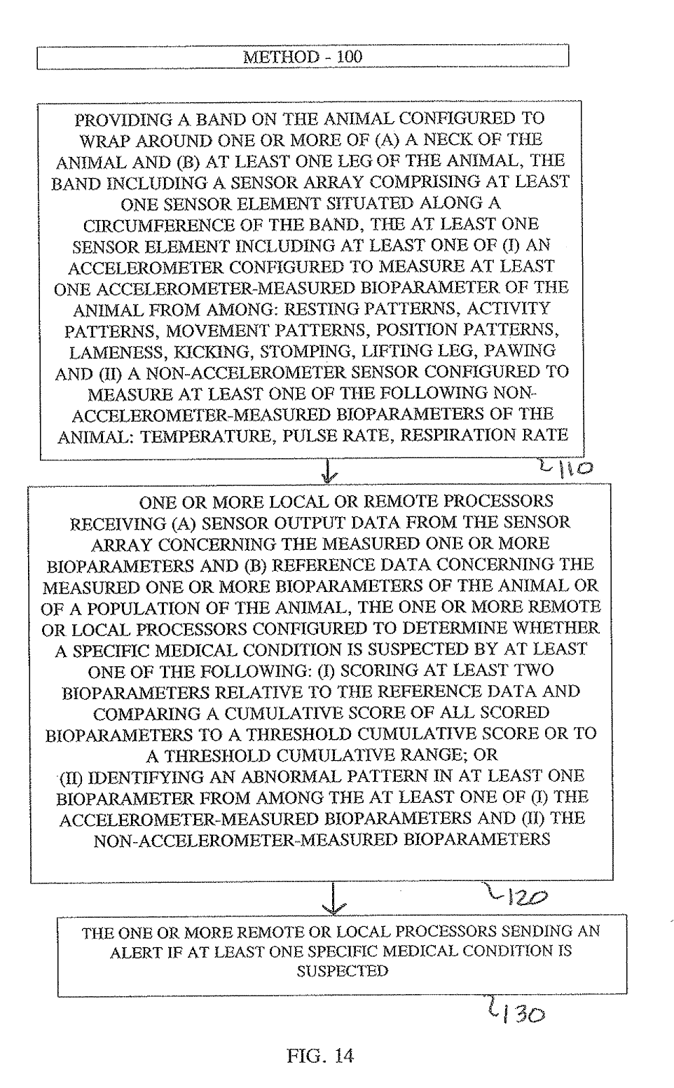

A sensor array (see FIG. 10) may include an acoustic sensor element 30e (piezoelectric element) for measuring pulse (heart rate) and an acoustic sensor for measuring respiration rate. Such a sensor array may include an accelerometer 30a to measure movement and vibrations of air traveling through the animal's air canals during inhaling and exhaling motions as well as the movement of blood traveling through the main blood vessels across the animal's neck. As shown in FIG. 10, the sensor array may also include a surface temperature/skin temperature sensor 30b to measure the surface temperature of the animal's body and an ambient temperature sensor 30f to measure the ambient temperature.

A sensor array may also include a microphone 30c. A sensor array may further include a microphone to listen to special noises made by an animal, for example a horse. Accordingly, the sounds picked up by a microphone may be interpreted by one ore more local processors 40 having an associated memory storage 67 (FIG. 10) of collar 20 or by one or more remote processors 40A of a remote computer terminal 69 (FIG. 11) and/or by a processor, such as at a server 70, having access to a dedicated or remote database to determine the type of sound and its interdependence with other vital sign bioparameters in order to arrive at a tentative diagnosis, to determine whether an alert is justified or to suggest treatment.

The sensor array 30 may also include a gyroscope 30d for capturing the vertical and/or horizontal movement of the animal. In the case of horses, there are numerous basic postures that provide information as to what the horse is doing and thereby assist in interpreting vital sign measurements to arrive at a tentative diagnosis. The following basic horse postures that may be detected by sensor elements 30, for example a gyroscope, an accelerometer and/or a magnetometer: lying down, lying on back, shifting weight, pawing, stomping, kicking, thrashing, turning head around for biting at flanks, standing on back legs, jumping, trotting, running, eating/drinking, limping hind leg, limping front leg, head bobbing, turning to lick, and stretching. The processor 40 make receive this information from the sensors 30 and utilize it in reaching a conclusion that it transmits remotely to the appropriate destination.

Each of the sensors 30 may be activated, de-activated, fine-tuned, set for predetermined repeated intervals or otherwise calibrated or controlled remotely, and in some embodiments also manually by a person located at the collar 20. “Remotely” means remote from the collar 20 and may include by a person in a vital sign monitoring station or a remotely stationed veterinarian or a medical center or the owner or the authorities or any other suitable location.

Band 20 may further include a remotely-actuatable speaker (not shown) for communicating sounds to the animal remotely and may include a remotely actuatable light (such as an LED or other light source) for illuminating the animal to those seeking to locate it. The speaker and light may also be actuatable manually in person. The speaker and light may be situated on or attached to the band 20 and may be included in a sensor array (even though the light is not a sensor).

As seen from FIG. 3, band 20 may also include a controller 49 that includes a local processor 40 that may be affixed to the collar 20 for example in a housing (not shown) attached to the band 20. As shown in FIG. 3, local processor 40 may also include a processing unit having MicroElectro Mechanical Systems (“MEMS”) technology. As also shown from FIG. 3, local processor 40 may be hard-wired or otherwise in electronic communication with each of the sensor elements 30. One or more local or remote processors may be configured to receive a signal representing data sensed by one or more of the sensor elements 30 and may be configured to analyze the data and communicate vital sign determinations and other data to a telecommunications system. The vital sign data measured by the sensor elements 30 of collar 20 may be relayed to and interpreted by processor 40 or by a remote processor (not shown). One or more local processors 40 or remote processors 40A may execute algorithms to interpret a collection of the physiological data sensed by the sensor elements and the interdependence of the vital sign data from the sensor elements and may arrive at a tentative diagnosis. The vital sign data may also include physiological data such as data about the movement of the animal (or other physiological data such, as the saltiness of the animal's skin) since this physiological data, when combined with fundamental vital signs such as breathing rate, respiration rate, pulse, temperature, etc. may be useful in diagnosis by the veterinarian or remote computer server for the automatic temporary diagnosis by the processor 40.

Controller 49 may also include a memory storage 67 for storing health information history of the animal, the memory storage accessible by the processor 40. The memory storage can be a flash memory or other memory storage devices known in the art.

As shown in FIG. 10, band 20 may include a communication device 68 such as a wireless transmitter unit, that may be accompanied by a receiving unit 68a forming a two-way communication device for communication to a remote station which may include a computer server pre-programmed to interact with the processor 40 or the remote station may communication with or include a veterinarian who can remotely measure vital signs using the collar's processor to select particular sensor elements to be activated to measure vital signs of the animal. As shown in FIG. 11, there is an option for there to be a remote station 70 (which may be a remote computer server having a remote processor 40A) which may also alert an animal owner or the authorities by sending an email communication 90a (FIG. 11) or an SMS alert 90b (FIG. 11). The communication device may also incorporate short range or long range wireless communication technology such as UHF, Wi-Fi, Bluetooth, etc. and cellular technology.

The band 20 and/or server computer or other part of the system such as the one or more local or remote processors may issue an alert based on predefined parameters (e.g. unique prior knowledge regarding the specific animal) and/or behavioral (e.g. erratic or uncharacteristic movements) or vital signs parameters. The specific measurements of the animal (height, length, weight etc.) and relevant history, as well of the population of animals of that species or breed or type, may be loaded into the device and/or the system during a registration procedure. The unique identification data of the animal can also include: the animal's name, owner's names, trainer's names, personal details (address, phone number etc.), medical information concerning the animal and any other relevant data. The information may be included in the processing by the one or more local or remote processors 40, 40A when the one or more local or remote processors 40, 40A analyze data from the sensor elements 30.

A GPS device may be incorporated into band or collar 20. The OPS device could take the form, for example, of an integrated circuit or an RFID. Other location awareness technology may also be incorporated into the band 20.

The receiving unit 68 attached to or incorporated into the band 20 may be a smart phone, mobile (and/or hand-held) device, or any other communication/messaging device, or a specifically designed receiver or reader. The receiving unit 68 may be connected to the band or collar 20 in a wired and/or wireless manner as mentioned above. The receiving unit 68 may be detachable from the band 20 for direct connection to a computer terminal, in order to enable faster or more secure downloading of stored (and in some cases processed) sensor data.

The band 20 and/or system may gather analytical information including statistics, trend analysis, comparative analysis etc. regarding particular horses, particular breeds of horses or other species of animals from the Equidae family. The system may incorporate a social network for other animal owners or trainers for the purpose of sharing information.

The vital sign and/or other physiological data acquired from sensor elements 30 may be further combined with information from other sensor elements 30 such as temperature, respiration rate and pulse and other available data such as the time of day, the ambient temperature, the animal's normal behavior, the context etc. The processor 40 may reach conclusions about the presence of a high probability of medical conditions suffered by the animal. If the sensor input indicates decreased or change in activity relative to the time of day and sounds of pain, an alert may be transmitted.

A method may also include, in some embodiments, a step of transmitting vital sign measurements to the animal owner, a veterinarian, a remote computer server or the authorities when the vital sign measurement exceeds a threshold level. Accordingly, processor 40 may be programmed to compare data received from the sensor elements to threshold levels of respiration rate, heart rate, temperature, movement, blood pressure, and/or other physiological data, such as noises made by a horse. Furthermore, the processor may have access to software in controller 49 that utilizes a function or a formula to relate combinations of the sensor element data. For example, if a horse moves in a certain way and utters a certain noise, that may trigger a particular alert or diagnosis. In addition, the programmer 40 may have access to its own data comparing the physiological data of a particular vital sign or combination of vital signs to the average vital sign data for horses or other species of the Equidae family, taking into consideration that breed and that geographical location, and taking into consideration the ambient temperature and the medical history of the animal. The controller/processor may transmit an alert to the animal owner and/or trainer, to a veterinarian or to the authorities.

A processor 40 affixed to the collar 20 may be in electronic communication with each of the at least two, or at least three or at least four sensor elements. The processor 40 may control a timing of an “ON” status of each sensor sufficient to trigger taking of a vital sign measurement. Memory storage 67 (FIG. 3) may be flash memory or other well known types of memory storage accessible by processor 40. The memory storage unit 67 may store data regarding the power requirements of each of the sensor elements in sensor array 30 as well as the lifespan of the battery 61 or other power source in collar 20. Alternatively, this data may be accessible by the processor 40 since processor 40 may be in communication with remote databases. As a result, the processor 40 may be configured to calculate the timing of the “ON” status of a sensor element (or of two or more or all the sensor elements) based on power requirements of the at least four sensors and a lifespan of the power source. In addition, processor 40 may receive sensor data from the sensor elements and communicate vital sign status of the animal to a remote location. The processor 40 may reach overall conclusions as to whether the animal has a particular medical condition by accessing databases and utilizing software containing diagnostic algorithms.

As seen from FIG. 13, the present invention may include a method 100 of monitoring an animal of the equidae family, for example a horse, to determine a suspicion of a specific medical condition in the animal. Method 100 may comprise a step 110 of providing a band on the animal configured to wrap around one or more of (a) a neck of the animal and (b) at least one leg of the animal, the band including a sensor array comprising at least one sensor element situated along a circumference of the band, the at least one sensor element including at least one of (i) an accelerometer configured to measure at least one accelerometer-measured bioparameter of the animal from among: resting patterns, activity patterns, movement patterns, position patterns, lameness, kicking, stomping, lifting leg, pawing and (ii) a non-accelerometer sensor configured to measure at least one of the following non-accelerometer-measured bioparameters of the animal: temperature, pulse rate, respiration rate.

Method 100 may also include a step 120 of one or more local or remote processors receiving (a) sensor output data from the sensor array concerning the measured one or more bioparameters and (b) reference data concerning the measured one or more bioparameters of the animal or of a population of the animal, the one or more remote or local processors configured to determine whether a specific medical condition is suspected by at least one of the following:

(i) scoring at least two bioparameters relative to the reference data and comparing a cumulative score of all scored bioparameters to a threshold cumulative score or to a threshold cumulative range; or

(ii) identifying an abnormal pattern in at least one bioparameter from among the at least one of (i) the accelerometer-measured bioparameters and (ii) the non-accelerometer-measured bioparameters.

Method 100 may include a step 130 of the one or more remote or local processors sending an alert if at least one specific medical condition is suspected.

In some versions of method 100, there may be a step of configuring the band with at least one acoustic concentrator and at least one acoustic balancer so as to reduce signal to noise ratio. There may also be a step of requiring the sensor output data from the sensor array concerning the bioparameters to pass a quality assurance test, the quality assurance test being based on a threshold level of signal to noise ratio.

Particular features described in the context of one embodiment may be able to be incorporated into other embodiments for which that feature was not specifically mentioned.

This patent application hereby incorporates by reference in its entirety all of the following published applications of Applicant: (i) Applicant's previously filed U.S. patent application Ser. Nos. 14/156,526 filed Jan. 16, 2014, having publication no. 2014-0123912 published May 8, 2014 having the Title “Pet Animal Collar for Health and Vital Signs Monitoring, Alert & Diagnosis”, which is a continuation in part of (ii) Applicant's U.S. Patent Application having Publication No. 20130014706 published Jan. 17, 2013 having the Title “Pet Animal Collar for Health and Vital Signs Monitoring, Alert & Diagnosis” and having a filing date of Feb. 21, 2012 and of (iii) Applicant's previously filed U.S. patent application Ser. No. 13/743,383 having the Title “Acoustically Enhanced Pet Animal Collar for Health & Vital Signs Monitoring, Alert and Diagnosis” filed Jan. 17, 2013 and published Jul. 17, 2014 under publication no. US2014-0196673A1.

The following lists certain examples of data sensed by an accelerometer, and certain examples of data sensed by an accelerometer combined data sensed by other sensors, which may be utilized in certain preferred embodiments of the present invention.

### For Accelerometer Only

Resting Patterns in an Adult Horse
Description—standing motionless or occasional leg shifting, infrequently lying down, mostly on the side (lateral recumbency), or on their sternum (ventral recumbency) and rarely on their back (dorsal recumbency). It is unknown exactly how much time is spent resting in an average horse. It is likely that this figure varies greatly between different individuals and influenced by various factors, such as age, breed, training, presence of other people/animals, health condition and lifestyle.
Normal range—determined by the system individually for each animal, which requires a short learning period.
Mostly motionless, except for short breaks of minimal movements for changing posture, location, looking around briefly, etc.
Includes breakdown of time spent in each: left or right lateral, dorsal or ventral recumbency.
Includes breakdown per hour of day and day of the week (to “learn” the animal's individual routine and lifestyle).
Sensory input—
Accelerometer—
Resting in any one posture with occasional switching.
Relatively short breaks of activity or exercise.
Alerts—
Detect trends of small changes over relatively long periods of time in overall time spent resting.
Detect significant changes over relatively short periods of time in overall time spent resting.
Detect trends of small changes over relatively long periods of time in overall time spent in each posture.
Detect significant changes over relatively short periods of time in overall time spent in each posture.
Detect changes over time in specific properties of each posture.

Activity Patterns in an Adult Horse
Description—horses exercise habits vary based on age, breed, training routine, presence of other people/animals, health condition, owner preference, housing conditions and lifestyle.
Normal range—determined by the system individually for each animal, which requires a short learning period.
Different types of activity patterns for each horse—walking, trotting, running, jumping etc.
Includes breakdown per hour of day and day of the week (to “learn” the horse's individual routine and lifestyle).
Sensory input—
Accelerometer—exercise, activity, lack of resting. The data is complementing the resting use case.
Alerts—
Detect persistent changes in specific properties of each activity.

Lameness
Description—abnormal gait is always an indication of pathology. It may result from a problem in one or more legs, vertebral column or nervous system.
Normal range—regular pattern of movements and regular level of activity as determined for this individual, which will require a short learning period.
Sensory input—
Accelerometer—
   Alterations in patterns of movement—lying down, sitting, walking and running
   Decreased overall level of activity
   Commonly, the problem starts during, or right after, exercise.
   Alterations may appear only during enhanced activity
   Alterations may appear after prolonged rest and get better with activity (or “warming up”).
Alerts—Detect persistent movement abnormalities

While the invention has been described with respect to a limited number of embodiments, it will be appreciated that many variations, modifications and other applications of the invention may be made. Therefore, the claimed invention as recited in the claims that follow is not limited to the embodiments described herein.

What is claimed is:

1. A system for monitoring vital signs of an animal of a Equidae comprising:
a band having a working surface configured to wrap around one or more of (a) a neck of the animal and (b) at least one leg of the animal;
a sensor array comprising at least one sensor element situated along a circumference of the band, the at least one sensor element including at least one of (i) an accelerometer configured to measure at least one accelerometer-measured bioparameter of the animal from among: resting patterns, activity patterns, movement patterns, position patterns, lameness, kicking, stomping, lifting leg, pawing and (ii) a non-accelerometer sensor configured to measure at least one of the following non-accelerometer-measured bioparameters of the animal: temperature, pulse rate, respiration rate;
one or more remote or local processors configured to receive (a) sensor output data from the sensor array concerning the measured one or more bioparameters and (b) reference data concerning the measured one or more bioparameters of the animal or of a population of the animal, the one or more remote or local processors configured to determine whether a specific medical condition is suspected by at least one of the following:
(i) scoring at least two bioparameters relative to the reference data and comparing a cumulative score of all scored bioparameters to a threshold cumulative score or to a threshold cumulative range; or
(ii) identifying an abnormal pattern in at least one bioparameter from among the at least one of (I) the accelerometer-measured bioparameters and (II) the non-accelerometer-measured bioparameters,
the one or more remote or local processors configured to send an alert if at least one specific medical condition is suspected.

2. The system of claim 1, wherein the at least one specific medical condition is colic and the at least one sensor element is a piezoelectric sensor configured to measure at least pulse rate.

3. The system of claim 1, wherein:
the sensor array comprises at least two sensor elements including the accelerometer and the non-accelerometer sensor;
the one or more remote or local processors are configured to receive (a) sensor output data from the sensor array concerning the measured bioparameters and (b) reference data concerning the measured bioparameters of the animal or of the population of the animal, the one or more remote or local processors configured to determine whether a specific medical condition is suspected by at least one of the following:
(i) scoring at least two bioparameters relative to the reference data and comparing a cumulative score of all scored bioparameters to a threshold cumulative score or to a threshold cumulative range; or
(ii) identifying an abnormal pattern in at least one bioparameter from among the accelerometer-measured bioparameters and the non-accelerometer-measured bioparameters.

4. The system of claim 3, further comprising combining the identifying of the abnormal pattern in the at least one bioparameter with identifying abnormal patterns in at least one other bioparameter.

5. The system of claim 3, wherein the identifying of the abnormal pattern in the at least one bioparameter involves identifying said abnormal patterns in at least one accelerometer-measured bioparameter and identifying abnormal patterns in at least one non-accelerometer-measured bioparameter.

6. The system of claim 5, wherein the specific medical condition is colic and the abnormal pattern in the at least one accelerometer-measured bioparameters is an abnormal pattern of movement comprising at least one of pawing; kicking; repeatedly lying down and rising; rolling or thrashing; change in activity level including lethargy or pacing or a constant shifting of weight when standing; stretching or abnormal posturing; or dorsal recumbency in foals sensed by the accelerometer.

7. The system of claim 6, wherein the non-accelerometer-measured bioparameters are at least one of elevated body temperature measured by a temperature sensor, change in the degree of gut sounds measured by a sound sensor, groaning measured by a sound sensor and loss of appetite measured by a proximity sensor.

8. The system of claim 3, wherein the sensor array is configured to measure at least two of the following specific medical conditions: colic, laminitis and lameness.

9. The system of claim 3, wherein the at least two sensor elements comprise at least four sensor elements distributed at different points along the circumference of the band.

10. The system of claim 3, wherein the one or more remote or local processors are configured to determine whether a new parameter, that is a function of a combination of each of the at least two bioparameters, exceeds a threshold level or range.

11. The system of claim 1, wherein the animal is a race horse, the at least one specific medical condition is fitness, the at least one sensor element is a piezoelectric sensor for measuring pulse, and wherein the one or more remote or local processors are configured to determine a heart rate variability from the pulse.

12. The system of claim 1, wherein the at least one specific medical condition is laminitis and the one or more remote or local processors are configured to base a suspicion of laminitis at least in part on
scoring at least two bioparameters from among (i) loss of appetite, (ii) decreased activity (iii) abnormal standing posture, (iv) walking with a slow, crouching, short-striding gait, (v) elevated skin temperature above a hoof and within 20 centimeters of the hoof of a leg of the animal, (vi) an exaggerated and bounding pulse in a leg of the animal, (vii) severe pain (viii) increased pulse rate (ix) increased respiratory rate, and (x) a an increased amount of time lying down,
and comparing a cumulative score of all scored bioparameters to a threshold cumulative score or to a threshold cumulative range.

13. The system of claim 1, wherein the at least one specific medical condition is lameness and the one or more remote or local processors are configured to
base a suspicion of lameness at least in part on
scoring the at least two bioparameters from among (i) shifting weight, (ii) abnormal positions, (iii) head bobbing, (iv) decreased activity, (v) pain, (vi) bounding pulses, wherein “(i)” through “(iv)” are measured by the accelerometer and “(v)” through “(vi)” are measured by a piezoelectric sensor.

14. The system of claim 1, further comprising a two-way communication device for communicating the suspicion in a form of an alert to a remotely stationed veterinarian or other user.

15. The system of claim 1, wherein the one or more remote or local processors are configured to determine whether each of the at least two bioparameters exceeds a threshold level or range.

16. The system of claim 1, wherein the band comprises a layer of an elastic material, the band also having a rear surface facing an opposite direction to the working surface.

17. The system of claim 1, wherein the at least one sensor is a piezoelectric sensor and further comprising at least one acoustic concentrator projecting toward a body portion of the animal from the working surface at one or more regions along the circumference; and at least one acoustic balancer projecting from the rear surface at the one or more regions along the circumference, the at least one acoustic balancer situated at a region along the circumference at least partly behind the at least one acoustic concentrators.

18. The system of claim 1, wherein the at least one sensor includes an accelerometer configured to measure at least two of pawing, kicking, rolling or thrashing, stretching.

19. The system of claim 1, wherein the animal is a horse and the band is configured to wrap around at least one leg of the horse above a hoof of said at least one leg, and within 20 centimeters of the hoof of the horse.

20. A system for monitoring vital signs of an animal of an Equidae family, comprising:
a band having a working surface configured to wrap around one or more of (a) a neck of the animal and (b) at least one leg of the animal;
one or more accelerometers situated along a circumference of the band and configured to measure at least one bioparameter of the animal from among: resting patterns, activity patterns, movement patterns, position patterns, lameness, kicking, stomping, lifting leg, pawing,
one or more remote or local processors configured to receive (a) sensor output data from the one or more accelerometers concerning the measured bioparameters and (b) reference data concerning the measured bioparameters of the animal or of a population of the animal, the one or more remote or local processors configured to determine whether a specific medical condition is suspected by at least one of the following:
(i) scoring at least two bioparameters and comparing a cumulative score of all scored bioparameters to a threshold cumulative score or to a threshold cumulative range; or
(ii) identifying an abnormal pattern in at least one accelerometer-measured bioparameter,
the one or more remote or local processors configured to send an alert if at least one specific medical condition is suspected.

21. The system of claim 20, further comprising combining the identifying of the abnormal pattern in the at least one bioparameter with identifying abnormal patterns in at least one other bioparameter.

22. The system of claim 20, wherein the one or more remote or local processors are configured to determine whether each of the at least two bioparameters exceeds a threshold level or range.

23. The system of claim 20, wherein the one or more remote or local processors are configured to determine whether a new parameter, that is a function of a combination of each of the at least two bioparameters, exceeds a threshold level or range.

24. A method of monitoring an animal of an Equidae family to determine a suspicion of a specific medical condition in the animal, comprising:
providing a band on the animal configured to wrap around one or more of (a) a neck of the animal and (b) at least one leg of the animal, the band including a sensor array comprising at least one sensor element situated along a circumference of the band, the at least one sensor element including at least one of (i) an accelerometer configured to measure at least one accelerometer-measured bioparameter of the animal from among: resting patterns, activity patterns, movement patterns, position patterns, lameness, kicking, stomping, lifting leg, pawing and (ii) a non-accelerometer sensor configured to measure at least one of the following non-accelerometer-measured bioparameters of the animal: temperature, pulse rate, respiration rate;
one or more local or remote processors receiving (a) sensor output data from the sensor array concerning the measured one or more bioparameters and (b) reference data concerning the measured one or more bioparameters of the animal or of a population of the animal, the one or more remote or local processors configured to determine whether a specific medical condition is suspected by at least one of the following:
(i) scoring at least two bioparameters relative to the reference data and comparing a cumulative score of all scored bioparameters to a threshold cumulative score or to a threshold cumulative range; or
(ii) identifying an abnormal pattern in at least one bioparameter from among the at least one of (i) the accelerometer-measured bioparameters and (ii) the non-accelerometer-measured bioparameters; and
the one or more remote or local processors sending an alert if at least one specific medical condition is suspected.

25. The method of claim 24, further comprising configuring the band with at least one acoustic concentrator and at least one acoustic balancer so as to reduce signal to noise ratio.

26. The method of claim 24, further comprising requiring the sensor output data from the sensor array concerning the bioparameters to pass a quality assurance test, the quality assurance test being based on a threshold level of signal to noise ratio.

27. The method of claim 24, wherein
the sensor array comprises at least two sensor elements including the accelerometer and the non-accelerometer sensor;
the one or more remote or local processors are configured to receive (a) sensor output data from the sensor array concerning the measured bioparameters and (b) reference data concerning the measured bioparameters of the animal or of the population of the animal, the one or more remote or local processors configured to determine whether a specific medical condition is suspected by at least one of the following:
(i) scoring at least two bioparameters relative to the reference data and comparing a cumulative score of all scored bioparameters to a threshold cumulative score or to a threshold cumulative range; or
(ii) identifying an abnormal pattern in at least one bioparameter from among the accelerometer-measured bioparameters and the non-accelerometer-measured bioparameters.
